(19) United States
(12) Patent Application Publication
(10) Pub. No.: US 2008/0152967 A1
(43) Pub. Date: Jun. 26, 2008

(54) PROCESS AND APPARATUS FOR PRODUCING HYDROGEN FROM SEWAGE SLUDGE

(76) Inventor: Sukomal Roychowdhury, San Diego, CA (US)

Correspondence Address: KRAMER LEVIN NAFTALIS & FRANKEL LLP;INTELLECTUAL PROPERTY DEPARTMENT, 1177 AVENUE OF THE AMERICAS, NEW YORK, NY, 10036, US

(21) Appl. No.: 11/960,655

(22) Filed: Dec. 19, 2007

Related U.S. Application Data
(60) Provisional application No. 60/871,877, filed on Dec. 26, 2006.

(51) Int. Cl. H01M 8/04; C12P 3/00; C12M 1/38

(52) U.S. Cl. 429/13; 435/168; 435/286.1

(57) ABSTRACT

A method of producing hydrogen from sewage sludge comprising: (a) introducing sewage sludge into an anaerobic digester containing a multiplicity of interfacing pairs of cathodes and anodes spaced from 0.5 to 1.5 inches from one another; (b) maintaining anaerobic conditions within said digester to cause the digestion of said sewage sludge; (c) applying an electric potential between said cathodes and anodes to create a current density of from 0.25 to 2.0 amps/square foot at said electrodes in order to suppress the formation of methane and enhance the production of hydrogen; and collecting gas containing hydrogen produced from said digester.

This application claims the benefit of U.S. Provisional Application No. 60/871,877 filed Dec. 26, 2006.

### BACKGROUND OF THE INVENTION

New sources of energy are needed to replace hydrocarbon-based fuels. Fossil fuels, particularly oil imported from foreign sources, are causing global warming, political problems and economic burdens.

Hydrogen is a fuel which does not produce pollutants, water being its only combustion product. Hydrogen also has many industrial uses in the production of fertilizers, dyes, drugs, plastics, hydrogenated oils and fats and other products.

### 1. FIELD OF THE INVENTION

This invention relates to a process for the production of hydrogen from anaerobically decomposed organic materials, in particular sewage sludge, by applying an electric potential to and thereby creating a current through the anaerobically decomposing sludge.

### 2. DESCRIPTION OF RELATED ART

The major established processes for producing hydrogen are: (1) steam reforming of natural gas, and (2) electrolysis of water.

Steam-reformation of natural gas is disadvantageous in that hydrocarbon fuels are consumed. Production of hydrogen by electrolysis of water, a relatively simple and non-polluting process, is costly and therefore economically disadvantageous for most industrial applications because the amount of energy needed for electrolysis of water exceeds the energy obtained from the combustion of the resulting hydrogen.

As disclosed in Roychowdhury, U.S. Pat. No. 6,090,266, unlike the methods for production of hydrogen outlined above, the process of the present invention does not depend on fossil fuels. The present process converts anaerobically decomposing organic materials, e.g., sewage sludge into hydrogen, while simultaneously reducing the time required to treat the sludge. The process of the invention uses a common, ubiquitous raw material found in sewage treatment plants, and, in one embodiment produces energy in the form of hydrogen.

In conventional sewage treatment plants, energy in the form of methane gas is produced from the anaerobic microbial digestion of organic matter. This is common in treatment facilities where sludge from primary treatment and secondary treatment is anaerobically digested in large vessels called digesters. The methane gas from the organic matter in the digesters is vented or flared, i.e. burned and thus ignored as a potential fuel. This is unfortunate because methane is a significant contributor to greenhouse gas.

About one-third of municipal wastewater treatment plants in the United States use digester gas to generate power on site. Some digester gas is used either directly to generate power in a power recovery turbine or indirectly to fuel boilers that produce steam or hot water to heat digesters. The balance is flared.

The process of anaerobic digestion occurs in three stages: (1) hydrolysis, (2) acidogenesis, and (3) methanogenesis.

In the hydrolysis step, enzymes in the sludge convert higher molecular weight organic compounds into compounds suitable for use as a source of energy. In acidogenesis, bacteria convert the compounds from the first stage into lower molecular weight intermediate compounds, e.g. hydrogen and carbon dioxide. In methanogenesis, methane creating bacteria convert the intermediate compounds into primarily methane and carbon dioxide.

Hydrogen and carbon dioxide are the principal building blocks of methane production. Bacteria that perform acidogenesis and methanogenesis, respectively, have a symbiotic or mutually beneficial relationship. The acidogenic bacteria [acidogens], in order to survive, must be in a reduced hydrogen environment. The methanogenic bacteria [methanogens], consume hydrogen and keep the concentrations of hydrogen low in the digester.

Conventional digesters in waste treatment facilities reduce volatile solids in sewage sludge by up to 55%. They generate substantial amounts of digester gas, primarily methane. The process typically takes from 21 days up to 32 days.

### OBJECTS OF THE INVENTION

It is an object of this invention to provide a method for producing hydrogen at low cost from anaerobically decomposing organic materials, e.g. sewage sludge.

It is a further and related object of this invention to provide a method of reducing the weight and volume of volatile solids in digested sewage sludge from waste treatment plants.

It is still a further object of this invention to provide a method for decreasing the time required to treat sewage sludge.

It is still a further object of the invention to provide an energy-efficient plant for the production of hydrogen from sewage sludge.

It is yet another object of the invention to provide an energy-efficient, retrofitted sewage treatment plant which is capable of producing hydrogen and concomitantly accelerating the treatment of the sewage sludge.

### SUMMARY OF THE INVENTION

The invention is in a process and apparatus for anaerobically treating sewage sludge emanating from the primary treatment stages of a sewage plant. The waste materials in anaerobically decomposing sewage sludge are processed in an anaerobic digester. The digester contains a multiplicity of interfacing arrays of cathodes and anodes. The spacing of the electrodes is set within a preferred range of 0.5-1.5 inches. A voltage is imposed across the sets of interfacing cathodes and anodes. A current density of 0.25 to 2.0 amps/square foot is maintained. The production of methane is suppressed and the production of hydrogen is promoted.

The hydrogen may be purified and then sold as a product; it may be passed into a fuel cell to generate electricity which is either returned to an external power grid or used in the digester, thereby reducing power costs; or, it may be fed into a turbine together with other digester gases to generate electricity which likewise is either returned to an external power grid or used in the digester, thereby reducing power costs.

In preferred digesters of the invention, the electrodes comprise 1.0-5.0 percent of the volume of the digester and the ratio of the effective area of the electrodes to the volume of the digester is from 3-30.

The invention is also in an improved sewage treatment plant which processes sewage sludge more than twice as fast as a conventional sewage treatment plant and produces substantially less methane. In this aspect of the invention, the advantage lies in the reduction of the treatment time and/or the more rapid digestion of volatile solids. This allows existing plants to process substantially more sewage with the production of little or no methane and allows new plants to be smaller and less expensive.

### THE DRAWINGS

FIG. 1 is a schematic flow sheet of the pilot plant in which the process of the invention has been performed in the apparatus of the invention.

FIG. 2 is a schematic flow sheet of an improved sewage treatment plant of the invention having multiple anaerobic digesters.

FIG. 3 is a further schematic flow sheet of an improved sewage treatment plant of the invention having multiple anaerobic digesters.

### DETAILED DESCRIPTION OF THE INVENTION

### Methods to Produce Hydrogen

In one embodiment of the invention hydrogen is produced from sewage sludge in a method comprising:
(a) introducing sewage sludge into an anaerobic digester containing a multiplicity of interfacing pairs of cathodes and anodes spaced from 0.5 to 1.5 inches from one another;
(b) maintaining anaerobic conditions within said digester to cause the digestion of said sewage sludge;
(c) applying an electric potential between said cathodes and anodes to create a current density of from 0.25 to 2.0 amps/square foot at said electrodes in order to suppress the formation of methane and enhance the production of hydrogen; and
(d) collecting gas containing hydrogen produced from said digester.

In other embodiments of the invention the current density is from 0.60-1.25 amps/square foot of electrode. In certain preferred embodiments, the current density is from 0.80-1.20 amps/square foot.

Desirably, the electrodes are spaced from one another by from 0.6-1.4 inches and preferably from 0.75-1.25 inches.

The temperature of the sludge can be maintained in the mesophilic range, 90-105° F. or in the thermophilic range, 110-135° F. Broadly, therefore, the temperature is maintained within the range of 90-135° F. Desirably it is maintained in the mesophilic range, 90-105° F. and preferably it is maintained in the range of 95-100° F.

The voltage across the pairs of electrodes is broadly from 1 to 8 volts, desirably from 2 to 5 volts and preferably about 3 volts.

Good results are obtained if the volume of the electrodes is broadly from 0.5 to 10 percent of the volume of the digester. Desirably the volume of the electrodes comprises 1.0 to 5.0 percent of the volume of the digester.

It is important to the satisfactory operation of the method that the electrodes have sufficient effective area exposed to the sewage sludge to convert the sludge to hydrogen in a period of time less than that required by conventional processes. By “effective area” is meant the area of the electrodes exposed to the sewage sludge. In order to achieve that end it is desirable to have the ratio of the effective area of the electrodes to the volume of the digester be broadly from 3 to 30 and desirably from 5 to 20. In a preferred embodiment the ratio is in the range from 7.5-12.5.

The methods of the invention can be carried out batchwise, semi-batchwise, i.e. where portions and portions of new sludge are added intermittently to the digester and processed sludge is removed intermittently from the digester. Or, the process may be carried out continuously.

The gas produced by the methods of the invention can be collected continuously from the digester or intermittently and either stored or passed to a purification system as is well known in the art. Desirably the hydrogen is purified by removal of other components, principally carbon dioxide.

### Methods to Accelerate the Treatment of Sewage Sludge and Reduce the Volume of the Solids in the Sewage Sludge

The invention also includes processes for accelerating the rate of digestion of sewage sludge and thereby reducing the time required to digest the sludge compared to conventional sewage treatment plants. The process conditions and the digester designs are essentially as disclosed above and below. It has been found that the treatment time of twenty-one days or more in conventional plants can be reduced to five to nine days when using the processes and apparatus of the invention. The invention achieves the same or higher reduction of the volume of volatile solids in the sludge in a much shorter time.

As further discussed herein, the term accelerating the rate of digestion of sewage sludge includes both conducting the anaerobic digestion in less time than a conventional sewage treatment plant and producing an effluent with the same reduced level of volatile solids, and, conducting the anaerobic digestion in the same time as a conventional sewage treatment plant and producing an effluent with less volatile solids than would otherwise be produced in a conventional plant in that same time period. As will be understood by those skilled in the art, some combination of the two advantages discussed above can be achieved as circumstances and economics dictate.

### The Digester and the Electrodes

The invention is also in a sewage sludge digester for producing hydrogen from sewage sludge and accelerating the rate of digestion of the sewage sludge. The digester comprises a sewage sludge digester designed, as known in the art to maintain anaerobic conditions in the sewage sludge contained therein, containing a multiplicity of interfacing cathodes and anodes distributed within the interior volume of the digester, the cathodes and anodes being spaced distant from each other by 0.5-1.5 inches. The total volume of the electrodes comprises broadly from 0.5 to 10 percent of the total volume of the digester. The ratio of the total effective surface area of the electrodes to the volume of the digester is broadly from 3 to 30 and desirably from 5 to 20. Good results are obtained if the ratio is from 7.5 to 12.

The digester of the invention includes a pump for introducing sewage sludge into said digester, a pump for removing processed sludge and a pump for recycling sludge within the digester to prevent settling of solids and ensure optimal processing of the sludge. The digester also includes a temperature control system, including a cooling system, for sensing and controlling the temperature of the sludge within said digester.

In addition, the digester includes an electrical control system for imposing a current density of 0.25-2.0 amps/square foot at the electrodes.

In a preferred embodiment the digester includes an electrical control system capable of imposing a voltage of from 2.0-5.0 volts across the pairs of electrodes and generating a current density between the pairs of electrodes of from 0.80-1.20 amps/square foot.

The anodes may be comprised of graphite and the cathodes may be comprised of aluminum or another corrosion-resistant material. The anodes and cathodes may be arranged in rows equidistant from one another such that both surfaces of each of the cathodes and anodes interface with an adjacent facing electrode of opposite polarity, except for the outside surfaces of the outside row of cathodes.

A preferred digester of the invention comprises a cylindrical sewage sludge digester containing a multiplicity of vertically-oriented, interfacing cathodes and anodes depending from horizontal racks. The cathodes and anodes are distributed in a plurality of equidistant, alternating rows of cathodes and anodes, respectively, within the interior volume of the digester and are being spaced from each other by 0.5 to 2.0 inches. The total volume of the electrodes comprises 1.0-5.0 percent of the total volume of said digester and the ratio of the total effective surface area of the electrodes to the volume of the digester is from 7.5 to 12.5. An electrical control system imposes a current density between pairs of cathodes and anodes of 0.25 to 2.0 amps/square foot.

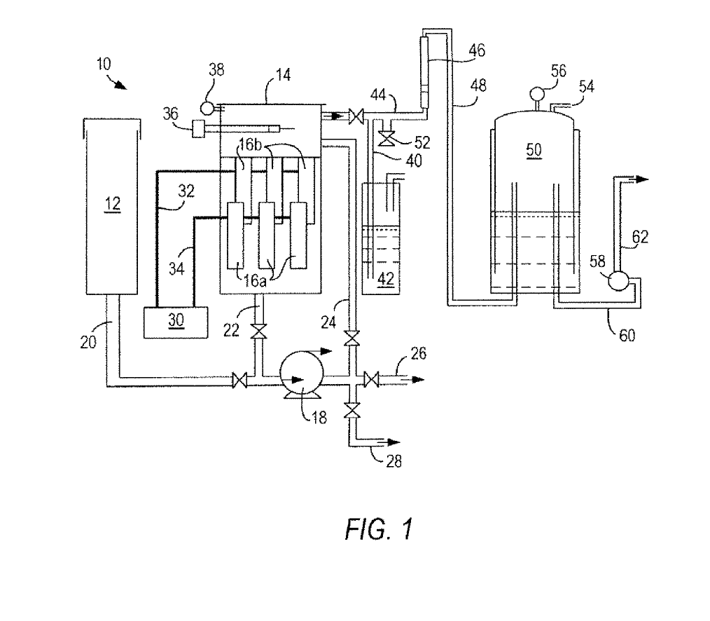

With reference to FIG. 1, reference numeral 10 refers to the pilot plant. Reference numeral 12 refers to a holding tank for sewage sludge and reference numeral 14 refers to the digester. Digester 14 is a 500-gallon cylindrical vessel. It has an internal diameter of 1.267 m, a straight side height of 1.87 m, a dished top and a shallow cone bottom.

Digester 14 contains electrodes. There are 57 electrodes, including 30 cathodes, 16a and 27 anodes, 16b. The length of each electrode is 1.41 m. Each electrode is 15.2± cm wide and 140.7 cm long. The anodes are comprised of graphite and are 1 cm thick. The cathodes are made of aluminum and are 0.13 cm thick. The anodes are arranged in nine rows of three and the cathodes are arranged in ten rows of three, so that the outside of the array is a cathode.

Following is a description of the several components.

| Component | Quantity | Specifications |
|---|---|---|
| Electrodes | 57 | 15.2 cm wide |
| Cathodes | 30 | 10 rows of 3 per row |
| Anodes | 27 | 9 rows of 3 per row |
| | Effective Surface | |
| Electrode | 2.3 sq. ft. | 140.7 cm. × 15.2 cm per face |
| Anode | 4.6 sq. ft. | 2 sides × 2.3 sq. ft. |
| Cathode | 4.6 sq. ft. | 2 sides × 2.3 sq. ft. |
| | Total Surface | |
| Cathodes | 11.5 sq. mt. | [(30 × 2)−6] × 2.3 sq. ft. (6 outside |
| | (124.2 sq. ft.) | cathode surfaces are not effective) |
| Anodes | 11.5 sq. mt. | 27 units × 2 sides × 2.3 sq. ft./unit = |
| | (124.2 sq. ft.) | 124.2 sq. ft. |
| Total volume of electrodes | 0.0652 m³ | |
| Total Digester Volume | 2.35 m³ | |
| Ratio of total volume of electrodes | 2.78% | |
| to total digester volume | | |
| Ratio of total effective area of | 9.78% | |
| electrodes to total digester volume | | |

The anodes and cathodes are spaced equidistant from one another about 1± inch apart.

The pilot plant includes pump 18 which serves the multiple purposes of feeding sewage sludge from holding tank 12 to digester 14 via line 20, recirculating sewage sludge within digester 14 via lines 22 and 24 and removing processed sludge via line 26 or 28 for sampling or removal, respectively. The valves in lines 20, 22, 24, 26 and 28 control the direction of flow.

A power supply unit is shown at reference numeral 30. The power supply unit is Power supply unit 30 imposes a voltage across the pairs of interfacing electrodes 16a and 16b via lines 32 and 34.

The pilot plant further includes temperature gauge 36 which measures the temperature of the sewage sludge in digester 14. A heater, not shown, can be deployed in holding tank 12 or feed line 20 to increase the temperature of the sewage sludge before it is fed to digester 14. A pressure gauge 38 measures the pressure of the gases in the upper part of digester 14.

The top portion of digester 14, where the generated gases collect, is connected via line 40 to a safety seal 42. The top portion of digester 14 is also connected via line 44 to rotameter 46 and then via line 48 to gas holder 50. The digester gas can be sampled at sample port 52 and the gas in gas holder 50 can be sampled at sample port 54. A pressure gauge is provided on gas holder 50 as shown at reference numeral 56.

Gas holder 50 has a floating head gas container in a water-filled vessel. The gas volume is measured by the changes in the level of the floating head gas container and continuously by rotameter 46.

A gas compressor 58 is provided to compress digester gas, supplied via line 60, to a gas purification unit, not shown, via line 62. The pilot plant includes amine scrubbers to purify the raw hydrogen gas and remove CO₂.

The composition of the product gas is sampled in a syringe, and analyzed in a gas chromatograph. The gas chromatograph includes a thermo conductivity detector and is equipped for analysis of H₂, O₂, N₂, methane, carbon monoxide, ethane, carbon dioxide, ethylene, NOx, acetylene, propane, butane, pentane, and C₆ through C₈ hydrocarbons. Nitrogen was used as the carrier gas.

Current meters and voltmeters were used to measure the voltage and amperage at each pair of electrodes. Direct current was used. Power consumption is based on regular measurements.

In the following Examples, pilot plant 10 was operated using sewage sludge as a feedstock. The unit was operated in batch mode. The sewage sludge was obtained from the sewage treatment plant in Bay Park, N.Y.

### EXAMPLE I

The cathodes were comprised of three 0.05-inch thick aluminum sheets spaced 4 mm+apart. The pilot plant was started up and steady state conditions were reached on the fourth day.

The concentration of hydrogen in the off-gas reached a high level of 73% with an average level of 62.8%. The gas mixture produced was monitored continuously. The following data summarize the operation.

| Total duration of run | 165 | hrs |
|---|---|---|
| Voltage across electrodes | between 3- | |
| | 3.2 volts | |
| Average Current | 137 | amps |
| Average Current Density | 1.1 | amps/ |
| | | sq. ft. |
| Spacing of electrodes | 1.5 | inch |

| | |
|---|---|
| Initial pH of sewage sludge | 7.75 |
| Average temperature in the digester | 103.2° F. |
| Peak concentration of hydrogen | 73% |
| Average concentration of hydrogen | 62.8% |

### EXAMPLE II

The cathodes comprised three 0.05-inch thick aluminum sheets bolted together leaving no space between the sheets. The pilot plant was started up and steady state conditions were reached on the fourth day. The concentration of hydrogen in the off-gas reached a high level of 72% by volume and an average level of 61.4%. The following data summarizes the operation.

| | | |
|---|---|---|
| Total duration of run | 212 | hrs |
| Voltage across electrodes | 3 | volts |
| Average Current | 134 | amps |
| Average Current Density | 1.07 | amps/ |
| | | sq. ft. |
| Spacing of electrodes | 1.5 | inch |

| | |
|---|---|
| Initial pH of sewage sludge | 7.8 |
| Average temperature in the digester | 107.3° F. |
| Peak concentration of hydrogen | 72.0% |
| Average concentration of hydrogen | 61.4% |

### EXAMPLE III

The cathodes comprised two 0.05-inch thick aluminum sheets bolted on either side of an 8 mm thick thermo-foam sheet. The pilot plant was started up and steady state conditions were reached on the fifth day. The average composition of the gas was 66.4% hydrogen.

After 141 hours, steady state conditions were reached. Fifteen gallons of digested sludge were then removed from the digester daily and replaced with 15 gallons of feed sludge in order to simulate the semi-batchwise operation of a sewage plant digester.

The following data summarize the operation.

| | | |
|---|---|---|
| Total duration of run | 141 | hrs |
| Voltage across electrodes | 3 | volts |
| Average Current | 129.5 | amps |
| Average Current Density | 1.04 | amps/ |
| | | sq. ft. |
| Spacing of electrodes | 1 | inch |

| | |
|---|---|
| Initial pH of sewage sludge | 7.8 |
| Average temperature of sewage | 103.7° F. |
| sludge within the digester | |
| Peak concentration of hydrogen | 71.6% |
| Average concentration of | 66.4% |
| hydrogen | |

The feed sludge contained 2.29% solids. After 141 hours, when steady state conditions were reached, the sludge contained 1.42% solids. The solids were thus reduced by about 38%. Solids content decreased as the process proceeded.

### SUMMARY OF EXAMPLES I, II, III

The results for steady state conditions are reported below.

| | | Percentage |
|---|---|---|
| Ex. | Volts | Hydrogen in Digester Gas |
| 1 | 3 | 65.8 |
| 2 | 3 | 61.4 |
| 3 | 3 | 66.4 |

The reduction of volatile solids content between the start and the end of the runs is 40% by weight over 7-9 days. Solids content decreased as the process proceeded.

### EXAMPLE IV

The process of the invention, as described in Examples I, II and III, is carried out under the following conditions.

| | | |
|---|---|---|
| Total duration of run | 220 | hrs |
| Voltage across electrodes | 5.0 | volts |
| Average Current | 175 | amps |
| Average Current Density | 1.41 | amps/sq. ft. |
| Spacing of electrodes | 1.25 | inch |

| | |
|---|---|
| Initial pH of sewage sludge | 7.7 |
| Average temperature of sewage | 105° F. |
| sludge within the digester | |

Satisfactory results are obtained.

### EXAMPLE V

The process of the invention, as described in Examples I, II and III, is carried out under the following conditions.

| | | |
|---|---|---|
| Total duration of run | 200 | hrs |
| Voltage across electrodes | 2.0 | volts |
| Average Current | 181 | amps |
| Average Current Density | 1.46 | amps/sq. ft. |
| Spacing of electrodes | 0.50 | inch |

| | |
|---|---|
| Initial pH of sewage sludge | 7.8 |
| Average temperature of sewage | 106° F. |
| sludge within the digester | |

Satisfactory results are obtained.

### EXAMPLE VI

The process of the invention, as described in Examples I, II and III, is carried out under the following conditions.

| | | |
|---|---|---|
| Total duration of run | 200 | hrs |
| Voltage across electrodes | 4.0 | volts |
| Average Current | 230 | amps |
| Average Current Density | 1.85 | amps/sq. ft. |
| Spacing of electrodes | 0.75 | inch |

| | |
|---|---|
| Initial pH of sewage sludge | 7.6 |
| Average temperature of sewage | 102° F. |
| sludge within the digester | |

Satisfactory results are obtained.

### EXAMPLE VII

The process of the invention, as described in Examples I, II and III, is carried out under the following conditions.

| | | |
|---|---|---|
| Total duration of run | 245 | hrs |
| Voltage across electrodes | 2.0 | volts |
| Average Current | 131 | amps |
| Average Current Density | 1.06 | amps/sq. ft. |
| Spacing of electrodes | 1.5 | inches |

| | |
|---|---|
| Initial pH of sewage sludge | 7.7 |
| Average temperature of sewage | 105° F. |
| sludge within the digester | |

Satisfactory results are obtained.

### EXAMPLE VIII

The process of the invention, as described in Examples I, II and III, is carried out under the following conditions.

| | | |
|---|---|---|
| Total duration of run | 300 | hrs |
| Voltage across electrodes | 2 | volts |
| Average Current | 58 | amps |
| Average Current Density | 0.47 | amps/sq. ft. |
| Spacing of electrodes | 1.5 | inches |

| | |
|---|---|
| Initial pH of sewage sludge | 7.5 |
| Average temperature of sewage | 106° F. |
| sludge within the digester | |

Satisfactory results are obtained.

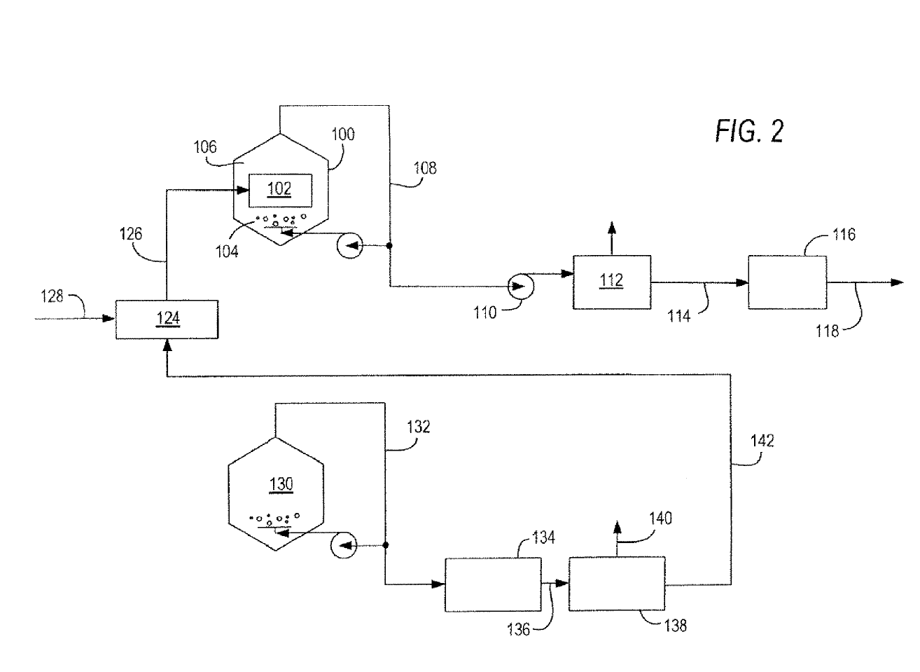

The process and apparatus of the invention can be used in stand alone plants for the production of hydrogen. Such a plant is depicted in FIG. 2.

With reference to FIG. 2, reference numeral 100 refers to a sewage sludge digester as described having a bank of electrodes, 102, also as described. Sewage sludge 104 is supplied to digester 100 which can operate in batch, modified-batch or continuous mode. Digester gas 106 produced in digester 100 passes via line 108 to compressor 110 where it is compressed and passes via line 112 to a conventional cleaning system. Cleaning system 112 may include

Purified hydrogen passes via line 114 to compressor 116 where it is compressed. Purified compressed hydrogen passes via line 118 to a distribution system and/or storage system not shown. Residual inert gases are vented from the system via line 120.

Electrical power for the process is supplied from power source 124 through conduit 126 to electrodes 102. The power supplied to power source 124 comes from an external source, e.g. power purchased from a public power utility grid, via conduit 128 and/or from power produced in turbines from gases, primarily methane, produced in a conventional anaerobic digester.

Reference numeral 130 depicts a conventional anaerobic digester. Digester 130 produces gases, including largely methane, which pass via line 132 to treatment and compression systems 134. The treated and compressed gas then passes via line 136 to one or more power turbines, 138. Exhaust gases are vented via line 140. The electrical output of power turbines 138 passes via conduit 142 to electrical power source 124.

The processes and apparatus of the invention can also be used in plants where the hydrogen produced in the digester is not sold as a product but rather is introduced to fuel cells to produce electric power. That power can then used in the process. By doing so, the demand for power from an external utility grid is reduced.

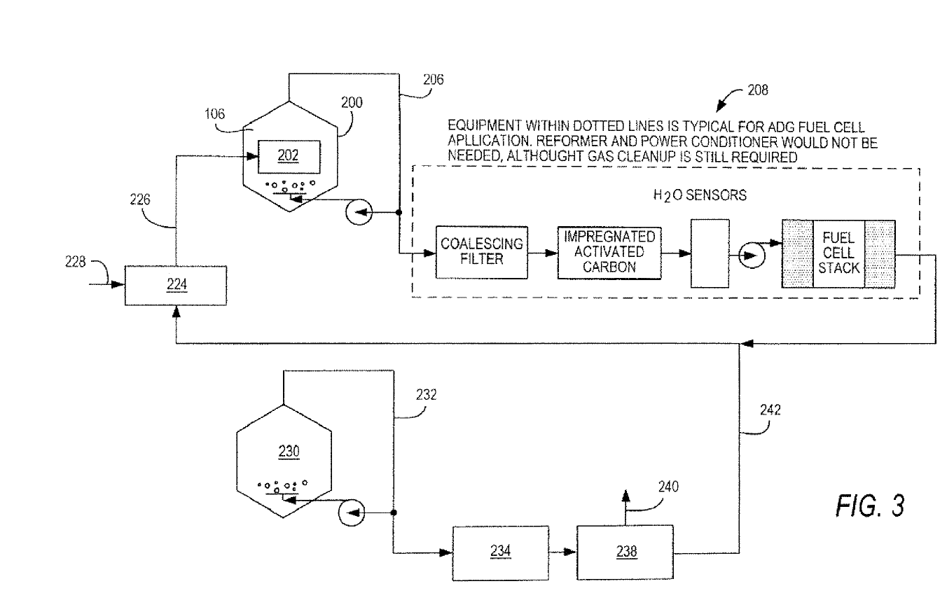

With reference to FIG. 3, reference numeral 200 refers to a sewage sludge digester as described having a bank of electrodes, 202, also as described. Sewage sludge 204 is supplied to digester 200 which can operate in batch, modified-batch or continuous mode.

Electrical power for the process is supplied from power source 224 through conduit 226 to electrodes 202. The power supplied to power source 224 comes from an external source, e.g. power purchased from a public power utility grid, via conduit 228 and/or from power produced in turbines from gases, primarily methane produced in a conventional anaerobic digester. Reference numeral 230 depicts a conventional anaerobic digester. Digester 230 produces gases, including largely methane, which pas via line 232 to treatment and compression systems 234. The treated and compressed gas then passes via line 236 to one or more power turbines, 238. Exhaust gases are vented via line 240. The electrical output of power turbines 238 passes via conduit 242 to electrical power source 224.

The gas produced in the digester passes via line 206 to a fuel cell system 208. Fuel cell system 208 is typical of those used to generate power from anaerobic digester gas. The hydrogen passes through coalescing filter 210 and then via line 212 to an impregnated active carbon filter 214 where impurities are removed. The purified gas then passes via line 216 to storage tank 218 and thence via line 220 to compressor 221. The compressed gas then passes via line 222 through fuel cell stack 223. The electric power generated in fuel cell stack 223 passes via conduit 225 to electrical power source 224 described above.

### Continuous and Semi Continuous Processes

It has been found that sewage treatment plant operation can be improved by the methods and apparatus of the invention, whether or not hydrogen is recovered as a product. Plants can be run in continuous and semi-continuous mode and treatment time can be reduced from twenty one or more days to five to nine days. The volatile solids can be reduced by 40% or more.

Introducing broadly from 5% to 20% new sludge relative to the digester volume, per day, desirably from 7.0% to 16.0% and most preferably from 9% to 13%, will result in significantly improved processing times as well as the production of hydrogen and suppression of methane. Stated otherwise, the space velocity of the sewage sludge may be broadly from 0.05-0.2/day desirably 0.07-0.16/day and preferably 0.09-0.13/day.

### Conversion of Other Organic Wastes to Hydrogen

The process and apparatus of the invention can also be applied to treat other organic waste materials and convert them to hydrogen gas by suppressing the methane generation. Such materials include animal wastes, agricultural waste, fruit and vegetable remnants in field and market places and cattle feed.

A suitable process is as follows. The waste materials are placed in an anaerobic vessel like a sewage treatment plant digester. The digester is fitted with heating, circulating and positive and negative electrodes. The waste materials are diluted with water to a 15-20% slurry. The temperature is kept between 95-100° F. As soon as the temperature of the slurry rises to within that range, 5-10% methane inoculum is thoroughly mixed with the contents of the digester. The inoculum is obtained from sewage sludge and landfill materials. The inoculum is added to expedite methane formation. Once methane generation reaches its maximum, 60-65%, the current is turned on. Gradually methane generation is suppressed and hydrogen production commences. Maximum production of hydrogen is reached when the off-gas comprises 70-75% hydrogen by volume.

What is claimed is:

1. A method of producing hydrogen from sewage sludge comprising:
(a) introducing sewage sludge into an anaerobic digester containing a multiplicity of interfacing pairs of cathodes and anodes spaced from 0.5 to 1.5 inches from one another;
(b) maintaining anaerobic conditions within said digester to cause the digestion of said sewage sludge;
(c) applying an electric potential between said cathodes and anodes to create a current density of from 0.25 to 2.0 amps/square foot at said electrodes in order to suppress the formation of methane and enhance the production of hydrogen;
(d) collecting gas containing hydrogen produced from said digester.

2. A method as recited in claim 1 wherein the said current density is from 0.60-1.25 amps/square foot.

3. A method as recited in claim 1 wherein the temperature is maintained between 80-130° F.

4. A method as recited in claim 1 wherein the said electrodes are separated by a distance of from 0.6-1.4 inches.

5. A method as recited in claim 1 wherein the said electrodes are separated by a distance of from 0.75-1.25 inches.

6. A method as recited in claim 1 wherein the voltage across said electrodes is from 2.0-5.0 volts.

7. A method as recited in claim 1 wherein sludge within said digester is recycled within said digester to prevent settling.

8. A method as recited in claim 1 wherein the temperature of the sewage sludge within the digester is sensed and steps are taken to maintain that temperature within the desired range.

9. A method as recited in claim 1 wherein the volume of the electrodes comprises from 0.5-10 percent of the volume of the interior of said digester.

10. A method as recited in claim 1 wherein the volume of the electrodes comprises from 1.0-5.0 percent of the volume of the interior of said digester.

11. A method as recited in claim 1 wherein the ratio of the total effective surface area of the electrodes to the total volume of the digester is from 3 to 30.

12. A method as recited in claim 11 wherein the ratio of the total effective surface area of the electrodes in the digester to the total volume of the digester is from 5 to 20.

13. A method as recited in claim 12 wherein the ratio is from 7.5-12.5.

14. A method as recited in claim 1 wherein the cathodes and anodes are disposed in rows spaced substantially equidistant from one another, both surfaces of each cathode and electrode facing an electrode of the opposite polarity, except for the outside rows of cathodes.

15. A method as recited in claim 1 wherein the cathode is comprised of aluminum.

16. A method as recited in claim 1 wherein the anodes are comprised of graphite.

17. A method as recited in claim 1 wherein the digester is a batch digester.

18. A continuous method as recited in claim 1 wherein a portion of fresh sewage sludge is introduced into the digester intermittently or continuously and wherein a portion of the processed sewage sludge is removed from the digester from time to time, intermittently or continuously.

19. A method as recited in claim 18 wherein the space velocity of the sewage sludge is from 0.05% to 0.20/day.

20. A method as recited in claim 1 wherein the hydrogen is purified and distributed as an end product.

21. A method as recited in claim 1 wherein the hydrogen is used to produce a power in a fuel cell.

22. A method as recited in claim 21 wherein the power produced in said fuel cell is used in the digester.

23. A method of producing hydrogen from sewage sludge and accelerating the treatment of said sewage sludge comprising the steps of:
(a) introducing a batch of sewage sludge into an anaerobic digester containing a multiplicity of interfacing pairs of cathodes and anodes distributed within the volume of said digester and spaced from 0.75-1.25 inches from one another;
(b) maintaining anaerobic conditions within said digester;
(c) applying an electric potential between said cathodes and anodes of from 2.0-5.0 volts to create a current density of from 0.60-1.25 amps/square foot to suppress the production of methane and enhance the production of hydrogen;
(d) collecting the off-gas from said digester;
(e) separating hydrogen from said off-gas; and
(f) removing processed sewage sludge from said digester.

24. A method as recited in claim 23 wherein the temperature within said anaerobic digesters is maintained at from 90-120° F.

25. A method as recited in claim 23 when the total volume of the electrodes within said digester comprises 1.0-5.0 percent of the interior volume of said digester and the ratio of the total effective area of the electrodes in the digester to the total volume of the digester is from 5 to 20.

26. A method as recited in claim 23 wherein the process is carried out continuously or semi-continuously and the space velocity of the sewage sludge is from 0.07-0.16/day.

27. A method as recited in claim 26 wherein the space velocity of the sewage sludge is from 0.09-0.13/day.

28. A sewage sludge digester for producing hydrogen from sewage sludge and concomitantly accelerating the rate of the digestion of sewage sludge, comprising:
(a) a sewage sludge digester adapted to maintain anaerobic conditions in the sewage sludge contained therein, said digester containing a multiplicity of interfacing cathodes and anodes distributed within the interior volume of said digester, said cathodes and anodes being spaced distant from each other by 0.75-1.25 inches, the total volume of said electrodes comprising 1.0-5.0 percent of the total volume of said digester and the ratio of the total effective surface area of said electrodes to the volume of said digester being from 5 to 20;
(b) means for introducing sewage sludge into said digester;
(c) gas effluent means for removing the gas produced within said digester;
(d) temperature control means for sensing and controlling the temperature of the sludge within said digester; and
(e) electrical control means for imposing a current density of 0.25-2.0 amps/square foot at said electrodes.

29. A digester as recited in claim 28 wherein the total volume of the electrodes within said digester comprises from percent of the total interior volume of said digester.

30. A digester as recited in claim 28 wherein the electrical control means is adapted to impose a voltage of from 2.0-5.0 volts across the pairs of electrodes and to generate a current density between said pairs of electrodes of from 0.80-1.20 amps/square foot.

31. A digester as recited in claim 30 further comprising means for recycling sludge within the digester to prevent settling.

32. A digester as recited in claim 30, wherein the said anodes are comprised of graphite and the said cathodes are comprised of aluminum and the anodes and cathodes are arranged in rows equidistant from one another such that both surfaces of each of said cathodes and anodes interface with an adjacent facing electrode of opposite polarity except for the outside surfaces of the outside row of cathodes.

33. A sewage sludge digester for producing hydrogen from sewage sludge and concomitantly accelerating the rate of the digestion of sewage sludge, comprising:
(a) a cylindrical sewage sludge digester adapted to maintain anaerobic conditions in the sewage sludge contained therein, said digester containing a multiplicity of vertically-oriented, interfacing cathodes and anodes depending from horizontal racks, said cathodes and anodes being distributed in a plurality of equidistant, alternating rows of cathodes and anodes, respectively, within the interior volume of said digester, said cathodes and anodes being spaced from each other by 0.75-3.0 inches, wherein the total volume of said electrodes comprises 1.0-5.0 percent of the total volume of said digester and the ratio of the total effective surface area of said electrodes to the volume of said digester is from 7.5 to 12.5;
(b) pumping means for introducing sewage sludge into said digester;
(c) gas effluent means for removing the gas produced within said digester;
(d) temperature control means for sensing and controlling the temperature of the sludge within said digester; and
(e) electrical control means for creating a current density between said pairs of cathodes and anodes of 0.80-1.20 amps/square foot.

34. A method of accelerating the anaerobic digestion of sewage sludge and reducing the amount of methane produced:
(a) introducing sewage sludge into an anaerobic digester containing a multiplicity of interfacing pairs of cathodes and anodes spaced from 0.5 to 1.5 inches from one another;
(b) maintaining anaerobic conditions within said digester to cause the digestion of said sewage sludge; and
(c) applying an electric potential between said cathodes and anodes to create a current density of from 0.25 to 2.0 amps/square foot at said electrodes in order to suppress the formation of methane and enhance the production of hydrogen;

35. A method as recited in claim 34 wherein the said current density is from 0.60-1.25 amps/square foot.

36. A method as recited in claim 34 wherein the temperature is maintained between 80-130° F.

37. A method as recited in claim 34 wherein the said electrodes are separated by a distance of from 0.6-1.4 inches.

38. A method as recited in claim 34 wherein the said electrodes are separated by a distance of from 0.75-1.25 inches.

39. A method as recited in claim 34 wherein the voltage across said electrodes is from 2.0-5.0 volts.

40. A method as recited in claim 34 wherein sludge within said digester is recycled within said digester to prevent settling.

41. A method as recited in claim 34 wherein the temperature of the sewage sludge within the digester is sensed and steps are taken to maintain that temperature within the desired range.

42. A method as recited in claim 34 wherein the volume of the electrodes comprises from 0.5-10 percent of the volume of the interior of said digester.

43. A method as recited in claim 34 wherein the volume of the electrodes comprises from 1.0-5.0 percent of the volume of the interior of said digester.

44. A method as recited in claim 34 wherein the ratio of the total effective surface area of the electrodes to the total volume of the digester is from 3 to 30.

45. A method as recited in claim 44 wherein the ratio of the total effective surface area of the electrodes in the digester to the total volume of the digester is from 5 to 20.

46. A method as recited in claim 44 wherein the ratio is from 7.5-12.5.

47. A method as recited in claim 44 wherein the cathodes and anodes are disposed in rows spaced substantially equidistant from one another, both surfaces of each cathode and electrode facing an electrode of the opposite polarity, except for the outside rows of cathodes.

48. A method as recited in claim 44 wherein the cathode is comprised of aluminum.

49. A method as recited in claim 44 wherein the anodes are comprised of graphite.

50. A method as recited in claim 44 wherein the digester is a batch digester.

51. A method as recited in claim 44 wherein a portion of fresh sewage sludge is introduced into the digester from time to time and wherein a portion of the processed sewage sludge is removed from the digester from time to time.

52. A method as recited in claim 44 wherein the hydrogen produced from the digester is separated from the other gases.

53. A method as recited in claim 44 wherein the hydrogen is purified and distributed as an end product.

54. A method as recited in claim 44 wherein the hydrogen is used to produce a power via a fuel cell.

55. A method as recited in claim 22 wherein the power produced in a fuel cell is used in the digester.

56. A batch-wise method of accelerating the anaerobic digestion of sewage sludge and reducing the amount of methane produced comprising the steps of:
(a) introducing a batch of sewage sludge into an anaerobic digester containing a multiplicity of interfacing pairs of cathodes and anodes distributed within the volume of said digester and spaced from 0.75-1.25 inches from one another;
(b) maintaining anaerobic conditions within said digester;
(c) applying an electric potential between said cathodes and anodes of from 2.0-5.0 volts to create a current density of from 0.60-1.25 amps/square foot to suppress the production of methane and enhance the production of hydrogen;
(d) collecting the off-gas from said digester;
(e) feeding said off-gas to a power generating turbine; and
(f) Using at least a portion of the power generated in said turbine in step (c); and
(g) removing processed sewage sludge from said digester.

57. A batch-wise method of accelerating the anaerobic digestion of sewage sludge and reducing the amount of methane produced comprising the steps of:
(a) introducing a batch of sewage sludge into an anaerobic digester containing a multiplicity of interfacing pairs of cathodes and anodes distributed within the volume of said digester and spaced from 0.75-1.25 inches from one another;
(b) maintaining anaerobic conditions within said digester;
(c) applying an electric potential between said cathodes and anodes of from 2.0-5.0 volts to create a current density of from 0.60-1.25 amps/square foot to suppress the production of methane and enhance the production of hydrogen;
(d) collecting the off-gas from said digester;
(e) feeding said off-gas to a power generating turbine; and
(f) using at least a portion of the power generated in said turbine in step (c); and
(g) removing processed sewage sludge from said digester.

58. A method as recited in claim 57 wherein the temperature within said anaerobic digesters is maintained at from 90-120° F.

59. A method as recited in claim 57 when the total volume of the electrodes within said digester comprises 1.0-5.0 percent of the interior volume of said digester and the ratio of the total effective area of the electrodes in the digester to the total volume of the digester is from 5 to 20.

60. A method as recited in claim 57 wherein the cathodes are comprised of aluminum and the anodes are comprised of graphite.

61. A method as recited in claim 57 wherein the sewage sludge is fed to the digester in a semi-continuous or continuous mode in which a portion of fresh sewage sludge is introduced into the digester from time to time or continuously and a portion of processed sewage sludge is removed from time to time or continuously from said digester.

62. A sewage sludge digester for accelerating the rate of the digestion of sewage sludge, comprising:
(a) a sewage sludge digester adapted to maintain anaerobic conditions in the sewage sludge contained therein, said digester containing a multiplicity of interfacing cathodes and anodes distributed within the interior volume of said digester, said cathodes and anodes being spaced distant from each other by 0.75-1.25 inches, the total volume of said electrodes comprising 1.0-5.0 percent of the total volume of said digester and the ratio of the total effective surface area of said electrodes to the volume of said digester being from 5 to 20;
(b) means for introducing sewage sludge into said digester;
(c) gas effluent means for removing the gas produced within said digester;
(d) temperature control means for sensing and controlling the temperature of the sludge within said digester; and
(e) electrical control means for imposing a current density of 0.25-2.0 amps/square foot at said electrodes.

63. A digester as recited in claim 62 wherein the total volume of the electrodes within said digester comprises from percent of the total interior volume of said digester.

64. A digester as recited in claim 62 wherein the electrical control means is adapted to impose a voltage of from 2.0-5.0 volts across the pairs of electrodes and to generate a current density between said pairs of electrodes of from 0.80-1.20 amps/square foot.

65. A digester as recited in claim 62 further comprising means for recycling sludge within the digester to prevent settling.

66. A digester as recited in claim 62, wherein the said anodes are comprised of graphite and the said cathodes are comprised of aluminum and the anodes and cathodes are arranged in rows equidistant from one another such that both surfaces of each of said cathodes and anodes interface with an adjacent facing electrode of opposite polarity except for the outside surfaces of the outside row of cathodes.

67. A sewage sludge digester for accelerating the rate of the digestion of sewage sludge, comprising:
(a) a cylindrical sewage sludge digester adapted to maintain anaerobic conditions in the sewage sludge contained therein, said digester containing a multiplicity of vertically-oriented, interfacing cathodes and anodes depending from horizontal racks, said cathodes and anodes being distributed in a plurality of equidistant, alternating rows of cathodes and anodes, respectively, within the interior volume of said digester, said cathodes and anodes being spaced from each other by 0.75-3.0 inches, wherein the total volume of said electrodes comprises 1.0-5.0 percent of the total volume of said digester and the ratio of the total effective surface area of said electrodes to the volume of said digester is from 7.5 to 12.5;
(b) pumping means for introducing sewage sludge into said digester;
(c) gas effluent means for removing the gas produced within said digester;
(d) temperature control means for sensing and controlling the temperature of the sludge within said digester; and
(e) electrical control means for creating a current density between said pairs of cathodes and anodes of 0.80-1.20 amps/square foot.

68. A method of anaerobically digesting sewage sludge comprising:
(a) continuously or intermittently introducing sewage sludge into an anaerobic digester containing a multiplicity of interfacing cathodes and anodes at a space velocity of from 0.05-0.2/day.
(b) maintaining anaerobic conditions within said digester to cause the digestion of said sewage sludge; and
(c) applying an electric potential between said cathodes and anodes of from 1.0-6.0 volts; and
(d) removing digested sewage sludge from said digester.

69. A method as recorded in claim 68 wherein said space velocity is from 0.07-0.16/day.

70. A method as recorded in claim 68 wherein said space velocity is from 0.09-0.14/day.
